(19) United States
(12) Patent Application Publication
(10) Pub. No.: US 2006/0278847 A1
(43) Pub. Date: Dec. 14, 2006

(54) Use of pulverulent mixtures containing hydrogen peroxide and hydrophobed silicon dioxide for the controlled release of hydrogen peroxide or oxygen

(75) Inventors: Egon Walzer, Maintal (DE); Corinna Schmidt, Steinau (DE); Friedhelm Brandner, Erlenbach (DE); Steffen Hasenzahl, Morris Plains, NJ (US); Mario Scholz, Grundau (DE); Birgit Del Grosso, Freigericht (DE)

Correspondence Address: FITCH, EVEN, TABIN & FLANNERY, P. O. BOX 18415, WASHINGTON, DC, 20036, US

(73) Assignee: Degussa AG, Dusseldorf (DE)

(21) Appl. No.: 11/284,399

(22) Filed: Nov. 18, 2005

Related U.S. Application Data
(63) Continuation of application No. PCT/EP04/04954, filed on May 10, 2004.

(30) Foreign Application Priority Data
May 23, 2003 (DE) 103 23 840.9
Jan. 15, 2004 (DE) 10 2004 002 356.5

(51) Int. Cl. C07C 409/00

(52) U.S. Cl. 252/186.23

(57) ABSTRACT

Pulverulent mixtures containing hydrogen peroxide and hydrophobised silicon dioxide are used for the controlled release of hydrogen peroxide and/or oxygen. The controlled release can take place in a time-delayed manner or by the application of pressure. Hydrogen peroxide can be released into aqueous or pulverulent media and in emulsions, gels, creams or pastes.

### CROSS REFERENCE TO RELATED APPLICATIONS

The present application is a continuation of international application PCT/EP2004/004954, which has an international filing date of May 10, 2004, and which was published in German under PCT Article 21(2) on Dec. 2, 2004. The international application claims priority under 35 U.S.C. § 119 to German applications 103 23 840.9, filed on May 23, 2003, and 10 2004 002 356.5, filed on Jan. 15, 2004, which are incorporated herein by reference in their entirety.

### FIELD OF THE INVENTION

The invention provides the use of pulverulent mixtures containing hydrogen peroxide and hydrophobised silicon dioxide for the controlled release of hydrogen peroxide and/or oxygen.

### BACKGROUND OF THE INVENTION

Hydrogen peroxide is produced and put onto the market in the form of aqueous solutions. For many applications it is advantageous to use a solid storage form of hydrogen peroxide instead of the aqueous solutions. Commercial solid storage forms for hydrogen peroxide are sodium carbonate perhydrate, sodium perborate and the adduct of urea with hydrogen peroxide. The disadvantage of these storage forms, however, is that together with hydrogen peroxide other water-soluble constituents are introduced, which are undesirable in many applications, for example because in aqueous solutions they lead to changes in the pH and can increase the salt content or the content of organic substances.

Pulverulent products are known from German laid-open specification DE 20 137 63 which contain hydrogen peroxide and at least 9 wt. % of a finely dispersed hydrophobised silica and which do not display the aforementioned disadvantage. As applications of these products, DE 20 137 63 cites bleaching, in particular of products containing oils, fats and cellulose, and addition to cleaning agents and cosmetic products. Apart from the property that dry powders are obtained with good stability, no other application-related properties are cited in DE 20 137 63.

In many applications in which hydrogen peroxide is used in the form of a solid storage form, it is also desirable for hydrogen peroxide to be released from the solid storage form in a controlled manner, e.g. by means of a delayed release over an extended period of time or a release in response to a changed physical variable.

### DESCRIPTION OF THE INVENTION

Surprisingly it has now been found that through the use of pulverulent mixtures containing hydrogen peroxide and hydrophobised silicon dioxide, a controlled release of hydrogen peroxide can be achieved in a simple manner without the release of other water-soluble substances. Through the use of these pulverulent mixtures, molecular oxygen can also be released instead of or together with hydrogen peroxide.

The invention thus provides the use of pulverulent mixtures containing hydrogen peroxide and hydrophobised silicon dioxide for the controlled release of hydrogen peroxide and/or oxygen.

Pulverulent mixtures are preferably used in which hydrogen peroxide is present in the form of drops of an aqueous solution of hydrogen peroxide, which are enclosed by hydrophobised silicon dioxide. Such pulverulent mixtures can be produced by simple means through the intensive mixing of an aqueous hydrogen peroxide solution with the hydrophobised silicon dioxide.

Hydrophobised silicon dioxides display non-polar organic groups at the surface and for that reason are not wetted by pure water. For the use according to the invention, hydrophobised silicon dioxides are preferably used which have been produced by reacting silicon dioxide powder with dimethyl dichlorosilane, octamethyl cyclotetrasiloxane, polydimethyl siloxane, octyl silane or hexamethyl disilazane.

A pyrogenically produced, hydrophobised silicon dioxide having a methanol wettability of at least 40 is preferably used as the hydrophobised silicon dioxide. Methanol wettability is a measure of the hydrophobicity of the silicon dioxide and is determined as the methanol content of a methanol-water mixture in percent by volume at which 50% of the hydrophobised silicon dioxide introduced into the methanol-water mixture forms a sediment. With a lower methanol content, wetting does not take place and most of the hydrophobised silicon dioxide floats. With a higher methanol content, extensive wetting takes place and the majority of the silicon dioxide forms a sediment. Through the use of pyrogenically produced, hydrophobised silicon dioxide having a methanol wettability of at least 40, pulverulent mixtures having a particularly good storage capacity are obtained which are in the form of free-flowing powders even with hydrophobised silicon dioxide contents of less than 9 wt. %.

Pulverulent mixtures are preferably used which contain hydrogen peroxide in the form of an aqueous solution with a content in the range from 5 to 70 wt. % of hydrogen peroxide and particularly preferably in the range from 10 to 50 wt. % of hydrogen peroxide. The aqueous hydrogen peroxide solution preferably contains one or more stabilisers, which stabilise the solution against decomposition to water and oxygen. Suitable stabilisers are, for example, stannates, phosphates, pyrophosphates, polyphosphates, organic phosphonic acids and aminophosphonic acids as well as salts thereof, nitrates and magnesium salts. Through the choice of the type and amount of stabilisers it is possible to influence whether it is predominantly hydrogen peroxide or predominantly oxygen which is released when the pulverulent mixtures are used.

In a preferred embodiment of the invention, the pulverulent mixtures containing hydrogen peroxide and hydrophobised silicon dioxide are used for the time-delayed release of hydrogen peroxide. The pulverulent mixtures are preferably exposed to only low pressures and shear forces for the time-delayed release of hydrogen peroxide. Oxygen can also be released in a time-delayed manner in place of hydrogen peroxide. Equally, both hydrogen peroxide and oxygen can be released simultaneously in a time-delayed manner.

The pulverulent mixtures are preferably used for the time-delayed release of hydrogen peroxide into an aqueous medium. To this end the pulverulent mixtures are brought into contact with the aqueous medium and optionally dispersed therein, dispersion preferably being performed with low shear forces. In a preferred embodiment the pulverulent mixture is brought into contact with the aqueous medium in a container which is permeable for water and hydrogen peroxide and impermeable for the hydrophobised silicon dioxide. The containers used for this purpose preferably consist entirely or partly of a filter medium whose pore size is smaller than the average particle size of the particles of hydrophobised silicon dioxide used to produce the pulverulent mixture. In one embodiment of the invention the container containing the pulverulent mixture is immersed in the aqueous medium such that transport of hydrogen peroxide out of the container and into the medium takes place largely by means of diffusion. In another embodiment the aqueous medium flows through the container. The containers can be of any shape and can take the form of a filter bag, a filter candle or a cartridge, for example. Through the use of such containers, hydrogen peroxide can be released in a time-delayed manner into an aqueous medium and at the same time the hydrophobised silicon dioxide left behind can be retained in the container.

Through the use according to the invention of pulverulent mixtures containing hydrogen peroxide and hydrophobised silicon dioxide for the time-delayed release of hydrogen peroxide into an aqueous medium, defined concentrations of hydrogen peroxide can be maintained in the aqueous medium for extended periods without the occurrence of undesired high peaks in the concentration of hydrogen peroxide and without the need for complex metering and regulating equipment. A use according to the invention of the pulverulent mixtures, wherein use is made of this advantage, is the time-delayed release of hydrogen peroxide in aquariums, for example, and in containers or ponds used for rearing fish. Through the use according to the invention of the pulverulent mixtures, a hydrogen peroxide concentration preventing multiplication of micro-organisms can be maintained without regulating equipment, without the development of damaging hydrogen peroxide concentrations for fish and fish larvae.

In a further embodiment of the invention, the pulverulent mixtures containing hydrogen peroxide and hydrophobised silicon dioxide are used to release hydrogen peroxide by the application of pressure. The pressure can be applied mechanically, by means of a plunger for example, hydraulically by means of liquid pressure, or pneumatically by means of gas pressure. Through the mechanical or pneumatic application of pressure hydrogen peroxide is released in the form of an aqueous solution which substantially has the same concentration as the hydrogen peroxide solution used to produce the pulverulent mixture. A hydraulic application of pressure is preferably used to release hydrogen peroxide into an aqueous medium. In this embodiment the pulverulent mixture is preferably used in a container which is permeable for water and hydrogen peroxide and impermeable for the hydrophobised silicon dioxide. The aqueous medium preferably flows through the container that is used, the pressure being applied by the dynamic pressure of the flow of aqueous medium. With this embodiment, hydrogen peroxide can be released into a flow of aqueous medium, the amount of hydrogen peroxide released being adjusted to the amount of the flow of medium, with no need for metering or regulating equipment.

Furthermore, the pulverulent mixtures containing hydrogen peroxide and hydrophobised silicon dioxide can also be used for the controlled release of hydrogen peroxide in a pulverulent medium. To this end the mixtures are mixed with the pulverulent medium, wherein an amount of the mixture containing hydrogen peroxide and silicon dioxide in the range from 0.1 to 10 wt. % of the total amount is preferably used. The mixtures containing hydrogen peroxide and hydrophobised silicon dioxide can be used to release hydrogen peroxide in the pulverulent medium in a time-delayed manner and so to maintain a low hydrogen peroxide concentration over an extended period of time. In addition, the free-flowing properties of the pulverulent medium can also be improved through the addition of the pulverulent mixture containing hydrogen peroxide and hydrophobised silicon dioxide. Furthermore, the mixtures containing hydrogen peroxide and hydrophobised silicon dioxide can also be used to release hydrogen peroxide in a pulverulent medium at a defined time through the application of pressure. The release of hydrogen peroxide through the application of pressure can be used, for example, to start a chemical reaction in the pulverulent medium, for example to cure the pulverulent medium.

The pulverulent mixtures containing hydrogen peroxide and hydrophobised silicon dioxide can also be added to emulsions, gels, creams or pastes for the controlled release of hydrogen peroxide and/or oxygen in the preparations thus obtained. The pulverulent mixtures can be used here for the time-delayed release of hydrogen peroxide in order to maintain a low concentration of hydrogen peroxide in the preparation for an extended period of time. Furthermore, the pulverulent mixtures can also be used to release hydrogen peroxide in the preparation at a defined time by the application of pressure or by the application of shear forces. The pulverulent mixtures are preferably used in cosmetic preparations to release hydrogen peroxide under the application of pressure through massaging onto the skin. The release of hydrogen peroxide through the application of pressure or the application of shear forces can also be used to trigger a chemical reaction in the preparation and thus to alter the composition and the properties of the preparation. Such preparations can be used as adhesives, for example, which crosslink and cure under the application of pressure.

Through the use of pulverulent mixtures containing hydrogen peroxide and hydrophobised silicon dioxide for the controlled release of hydrogen peroxide, in particular into an aqueous medium, a pulverulent medium, an emulsion, a gel, a cream or a paste, the multiplication of micro-organisms can be inhibited in the medium into which the hydrogen peroxide is released and even the killing of micro-organisms can be achieved if an effective amount is released. When used with a time-delayed release of hydrogen peroxide, hydrogen peroxide can be released over an extended period in an amount which inhibits the multiplication of micro-organisms and thus brings about the preservation of the medium into which the hydrogen peroxide is released. When used to release hydrogen peroxide under the application of pressure, hydrogen peroxide can be deliberately released at a particular moment by the application of pressure in an effective quantity to destroy micro-organisms and thus a preparation is deliberately given a disinfectant action at a specified time.

In one embodiment of the invention, the pulverulent mixtures containing hydrogen peroxide and hydrophobised silicon dioxide are used to produce wound treatment agents which when used develop a microbial growth-inhibiting and optionally also a disinfectant action through the release of hydrogen peroxide under the application of pressure. Such wound treatment agents can be realised as plasters or as creams, ointments or gels for wound treatment.

In a further embodiment of the invention, the pulverulent mixtures containing hydrogen peroxide and hydrophobised silicon dioxide can also be used for the controlled release of oxygen into a gaseous medium. The release of oxygen preferably takes place in a time-delayed manner such that a specific oxygen concentration is maintained in the gaseous medium over an extended period of time. Through the release of oxygen over an extended period of time, an adequate oxygen concentration can be maintained in the gaseous medium even when oxygen is withdrawn from the gaseous medium, for example through metabolic reactions of micro-organisms. By maintaining an adequate oxygen concentration, the growth of anaerobic micro-organisms can be inhibited and hence the formation of volatile metabolic products by such anaerobic micro-organisms can also be inhibited. The time-delayed release of oxygen from pulverulent mixtures containing hydrogen peroxide and hydrophobised silicon dioxide can thus also be used to prevent odour formation, the cause of which is the formation of volatile metabolic products by anaerobic micro-organisms. Examples of such a use according to the invention to prevent odour formation are the use in hygiene products which are worn on the body, in food packaging, in storage containers for rotting waste and in air filters.

### EXAMPLES

### Preparation of Pulverulent Mixtures Containing Hydrogen Peroxide and Hydrophobised Silicon Dioxide

In Example 1, 93 g of a 10 wt. % hydrogen peroxide solution were mixed with 7 g of Aerosil® R812S in a multimixer (Braun, model MX32) at the highest setting for 45 s. The high shear forces of the mixer reduce the liquid to small droplets, which are enclosed by the hydrophobic Aerosil. The mixture obtained is a free-flowing powder.

Examples 2 to 4 were performed in the same way with the amounts and concentrations of hydrogen peroxide and amounts and grades of hydrophobised Aerosil listed in Table 1 and likewise produced free-flowing powders.

TABLE 1
Preparation of pulverulent mixtures containing hydrogen
peroxide and hydrophobised silicon dioxide

Example

| | | | |
|---|---|---|---|
| 1 | 2 | 3 | 4 |

| | | | | |
|---|---|---|---|---|
| Amount of H₂O₂ solution in g | 93.0 | 93.0 | 93.0 | 95.0 |
| Concentration of H₂O₂ | 10.0 | 35.0 | 50.0 | 10.0 |
| solution in wt. % | | | | |
| Hydrophobised Aerosil | R812S | R812S | R812S | R202 |
| Methanol wettability | 60 | 60 | 60 | 70 |
| Amount of hydrophobised | 7.0 | 7.0 | 7.0 | 5.0 |
| Aerosil in g | | | | |

### Time-Delayed Release of Hydrogen Peroxide Into an Aqueous Medium

In Example 5, 4 g of the product obtained in Example 1 were introduced into a commercial teabag and the teabag was sealed. The teabag was completely submerged in 1 litre of demineralised water in a stirred vessel and the solution was stirred slowly at room temperature. Samples were taken at regular intervals and the hydrogen peroxide content of the solution determined by cerimetric titration.

Example 6 was performed in the same way with the product obtained in Example 2.

TABLE 2
Time-delayed release of H₂O₂ into an aqueous medium

H₂O₂ content in g/l

| Time in h | Example 5 | Example 6 |
|---|---|---|
| 0 | 0 | 0 |
| 2 | 0.02 | 0.08 |
| 4 | 0.03 | 0.17 |
| 6 | 0.06 | 0.52 |
| 16 | 0.08 | 0.77 |
| 24 | 0.10 | 1.03 |
| 48 | 0.17 | 1.08 |
| 72 | 0.23 | 1.17 |

The results summarised in Table 2 show that hydrogen peroxide was released from the teabags into the surrounding aqueous medium in a time-delayed manner over a period of over 48 h.

### Release of Hydrogen Peroxide Through the Application of Pressure

### EXAMPLE 7

10 g of the product obtained in Example 1 were subjected to an overpressure of 4 bar of compressed air in a pressure filter (pore size 1.2 μm). The hydrogen peroxide released under the application of pressure was collected in a reservoir. 33% of the hydrogen peroxide contained in the product had been collected in the form of an aqueous solution within 15 min and a total of 50% of the initial amount within 30 min.

### EXAMPLE 8

10 g of the product obtained in Example 1 were placed on a sintered glass filter (G2) and a water jet vacuum was applied to the filter. The hydrogen peroxide released by the atmospheric pressure in the form of an aqueous solution was collected in a receiver. 33% of the hydrogen peroxide contained in the material used had been released within 15 min and a total of 50% within 60 min.

### Time-Delayed Release of Hydrogen Peroxide Into a Pulverulent Medium

### EXAMPLE 9

165 g of a commercial edible starch were mixed with 1.8 g of the product obtained in Example 1. The starch treated in this way was stored in an open container for 13 weeks at 20 to 23° C. and a humidity of 50 to 60%. No infestation by mould or other micro-organisms was visible during storage. After storage the treated starch displayed no deterioration in its free-flowing properties.

After comparable storage an untreated sample of the same starch displayed clumping and the first signs of mould infestation.

### Time-Delayed Release of Oxygen

### EXAMPLE 10

The product obtained in Example 3 was stored at room temperature for a period of 60 days. During this time the hydrogen peroxide content of the powder fell from an initial 50.1 wt. % to 45.6 wt. % after 30 days and 40.5 wt. % after 60 days. The decomposition of hydrogen peroxide during storage released oxygen into the ambient air, whereby the amount of oxygen released is calculated at 15 Nl/kg after 30 days and 32 Nl/kg after 60 days (Nl=standard litres). Over a period of 60 days a uniform release of oxygen into the ambient air thus occurred at a rate of approx. 0.5 Nl/kg·d.

1-14. (canceled)

15. A method for the controlled release of hydrogen peroxide and/or oxygen, wherein hydrogen peroxide and/or oxygen is released from a pulverulent mixture comprising hydrogen peroxide and hydrophobised silicon dioxide.

16. The method of claim 15, wherein hydrogen peroxide is present in the pulverulent mixture in the form of drops of an aqueous solution of hydrogen peroxide enclosed by hydrophobised silicon dioxide.

17. The method of claim 15, wherein a pyrogenically produced, hydrophobised silicon dioxide having a methanol wettability of at least 40 is used as the hydrophobised silicon dioxide.

18. The method of claim 15, wherein the release of hydrogen peroxide and/or oxygen takes place in a time-delayed manner.

19. The method of claim 15, wherein hydrogen peroxide is released under the application of pressure.

20. The method of claim 15, wherein hydrogen peroxide is released into an aqueous medium.

21. The method of claim 20, wherein the pulverulent mixture is used in a container which is permeable for water and hydrogen peroxide and impermeable for the hydrophobised silicon dioxide.

22. The method of claim 15, wherein hydrogen peroxide is released into a pulverulent medium.

23. The method of claim 15, wherein the pulverulent mixture is added to an emulsion, a gel, a cream or a paste.

24. The method of claim 15, wherein hydrogen peroxide is released in an amount which inhibits the multiplication of micro-organisms.

25. The method of claim 15, wherein hydrogen peroxide is released in an effective amount to kill micro-organisms.

26. The method of claim 15, wherein oxygen is released into a gaseous medium.

27. The method of claim 15, wherein oxygen is released in an amount which inhibits the formation of volatile metabolic products by anaerobic micro-organisms.

28. The method of claim 15, wherein the pulverulent mixture is part of a wound treatment agent and wherein hydrogen peroxide is released during use of said wound treatment agent under the application of pressure in an amount which inhibits the multiplication of micro-organisms.
